(19) United States
(12) Patent Application Publication
(10) Pub. No.: US 2020/0129342 A1
(43) Pub. Date: Apr. 30, 2020

(54) DEVICE FOR VENTILATING AN ORTHOPEDIC CAST

(71) Applicant: Riley SHOVE, Manchester, CT (US)

(72) Inventor: Riley SHOVE, Manchester, CT (US)

(21) Appl. No.: 16/169,783

(22) Filed: Oct. 24, 2018

(51) Int. Cl. A61F 13/04; A61F 5/01

(52) U.S. Cl. CPC A61F 13/046; A61F 7/02; A61F 5/01

(57) ABSTRACT

Provided herein is a device for ventilating an orthopedic cast. The device includes (a) an air-pump; and (b) a flexible tubing connected to the air-pump; such that the flexible tubing is insertable into the space between the orthopedic cast and the body of the wearer of the orthopedic cast. The air-pump permits air to be forced into the flexible tubing to ventilate the space between the orthopedic cast and the body of the wearer of the orthopedic cast through an opening in the flexible tubing.

### TECHNICAL FIELD

The present invention relates to a ventilation system or device, which can be used to ventilate a rigid support system, such as an orthopedic cast.

### BACKGROUND

Orthopedic casts, or commonly referred to as casts are encasings that are used to rigidly support a limb or some other body-part of a wearer. They are often used when a limb, such as a leg or arm, is broken to hold the limb rigid while a broken bone or other injury heals. Sweat-related irritation is a common problem associated with a cast placed over broken limbs. This could be particularly problematic when casts are worn in warm weather or for long periods of time. Left unattended, it can cause itching, burns, and allergic contact dermatitis. In hot weather, staphylococcal infection of the hair follicles and sweat glands can lead to severe and painful dermatitis.

One way of addressing this issue is to use casts, which can be frequently removed and/or replaced. However, not all kinds of injuries can be healed with such casts and it can be expensive if multiple casts are need. Ventilation devices for casts are also available, which need to be connected to a vacuum pump, and can promote air-flow inside a cast by sucking the air using the pump. Such devices, however, need to be connected to a bulky vacuum cleaner, which typically operates by being connected to an AC outlet.

Thus, there is a need for a device that can not only ventilate a cast, but is also easily operable, and portable such that wearers of all age, including children and aged people can readily carry and use them. Preferably, such casts would not rely on being connected to an AC power source for its operation.

### SUMMARY

Provided herein is a system, device and method that addresses many of the disadvantages of prior art devices. As can be seen from the description below, the present invention provides a simple and effective ventilation system to ventilate a cast and remove moisture entrapped between the cast and the body of the wearer.

In one aspect, provided herein is a device for ventilating an orthopedic cast comprising: (a) an air-pump; and (b) a flexible tubing connected to the air-pump; wherein the flexible tubing is insertable into the space between the orthopedic cast and the body of the wearer of the orthopedic cast; wherein the air-pump permits air to be forced into the flexible tubing; and wherein the flexible tubing comprises an opening that permits the air to ventilate the space between the orthopedic cast and the body of the wearer of the orthopedic cast.

In another aspect, described herein is a device for ventilating an orthopedic cast comprising: a flexible tubing attached to the orthopedic cast and disposed in the space between the orthopedic cast and the body of the wearer of the orthopedic cast; wherein the flexible tubing is connectable to an air-pump that permits air to be forced into the flexible tubing; and wherein the flexible tubing comprises an opening that permits the air in the flexible tubing to ventilate the space between the orthopedic cast and the body of the wearer of the orthopedic cast.

Also provided is a method of ventilating an orthopedic cast comprising: (a) inserting a flexible tubing into the space between the orthopedic cast and the body of the wearer of the orthopedic cast, wherein the flexible tubing is connected to an air-pump and comprises an opening through which air can be released from the flexible tubing into the space between the orthopedic cast and the body of the wearer of the orthopedic cast; and, (b) operating the air-pump to release air into the flexible tubing, thereby ventilating the space between the orthopedic cast and the body of the wearer of the orthopedic cast.

The described device and method of ventilating orthopedic casts has several advantages including the ease of use, portability, and the fact that it does not need electricity to operate.

### BRIEF DESCRIPTION OF THE DRAWINGS

FIG. 1 provides an example of the device for ventilating an orthopedic cast, which contains one opening at the end of the tubing that permits ventilation of the orthopedic cast.

FIG. 2 provides an example of the device for ventilating an orthopedic cast, wherein the flexible tubing is attached to the cast such that it can wrap around the arm of the wearer.

FIG. 3 provides an example of the device for ventilating an orthopedic cast, which contains multiple openings in the tubing that permit ventilation of the orthopedic cast.

### DETAILED DESCRIPTION

In one aspect, provided herein is a device for ventilating an orthopedic cast comprising: (a) an air-pump; and (b) a flexible tubing connected to the air-pump; wherein the flexible tubing is insertable into the space between the orthopedic cast and the body of the wearer of the orthopedic cast; wherein the air-pump permits air to be forced into the flexible tubing; and wherein the flexible tubing comprises an opening that permits the air to ventilate the space between the orthopedic cast and the body of the wearer of the orthopedic cast.

In another aspect, described herein is a device for ventilating an orthopedic cast comprising a flexible tubing attached to the orthopedic cast. The attached flexible tubing is disposed in the space between the orthopedic cast and the body of the wearer of the orthopedic cast. The flexible tubing is connectable to an air-pump that permits air to be forced into the flexible tubing; and comprises an opening that permits the air in the flexible tubing to ventilate the space between the orthopedic cast and the body of the wearer of the orthopedic cast.

Also provided is a method of ventilating an orthopedic cast comprising: (a) inserting a flexible tubing into the space between the orthopedic cast and the body of the wearer of the orthopedic cast, wherein the flexible tubing is connected to an air-pump and comprises an opening through which air can be released from the flexible tubing into the space between the orthopedic cast and the body of the wearer of the orthopedic cast; and, (b) operating the air-pump to release air into the flexible tubing, thereby ventilating the space between the orthopedic cast and the body of the wearer of the orthopedic cast.

In some embodiments, the flexible tubing can run longitudinally along the length of the case. In some embodiments, the flexible tubing can run transversely in a helical or spiral manner along the cast.

In one embodiment, the flexible tubing of the device comprises more than one opening that permits the air to be released into the space between the orthopedic cast and the body of the wearer of the orthopedic cast. The number of openings could be adjusted depending on the length of the flexible tubing and the desired air-pressure. For example, the flexible tubing could have 2, 3, 4, 5, 6, 7, 8, 9, 10 or more openings.

In one embodiment, the flexible tubing of the device is connected to the air-pump via a connector. The connector could be of suitable structure and material. For example, it can be made of another tubing of a diameter larger than the flexible tubing. The connector may be made of materials such as plastic, rubber or metal.

In one embodiment, the air-pump connected to the device can be operated manually. In one embodiment, the air-pump can be operated using electric energy. The electric energy could be provided by a battery, or by connecting the device to any other DC electric power source. In some embodiments, the air-pump permits adjusting the pressure of the air forced into the flexible tubing.

The flexible tubing of the device can be made of any suitable material such as polyolefin, polyethylene, polypropylene, nylon, rubber, silicone, neoprene or latex. For example, in one embodiment, the flexible material is made of biocompatible material. The biocompatible material could be any biocompatible such as biocompatible plastic or rubber. In some embodiments, the flexible tubing is cylindrical in shape. In some embodiments, it is flat in shape. In some embodiments, the flexible tubing is flat when the air-pump is not being operated but inflates when the air-pump is being operated.

The flexible tubing can have a diameter, for example, of about 1-0.5 inch, about 0.5-0.1 inch, or about 0.2-0.3 inch, or about 0.1-0.05 inch. In some embodiment, the flexible tubing can have a diameter of about 0.25 inch.

In some embodiments, the device is attached to the orthopedic cast, while in some embodiments, it is not attached to the orthopedic cast. For example, the device can be attached to the orthopedic cast by attaching the flexible tubing and/or the air-pump to the cast by adhesive, clip or Velcro or any suitable attaching means. In some embodiments, the device comes with an attachment means that allows easy attachment and detachment, for example, by means of a Velcro strap.

An orthopedic cast as used herein, also referred to as a cast, is a shell, encasing a limb (or, in some cases, larger portions of the body) to stabilize and hold anatomical structures, most often a broken bone (or bones), in place until healing is confirmed. It can be made of any suitable material, including plaster, calcined gypsum, bandage or fiberglass.

An air-pump can be any pump that can be a source of pressurized air, including a bulb pump, a balloon pump, etc. Such pumps could include a means for operating the valve manually, such as by squeezing the bulb of a bulb pump with a hand. The air pressure can be controlled by valves such as a push-button air release valve, a turn-type airflow control valve. Any air compartments or bulbs associated with such pumps could have a volume of about 1 in³, about 2 in³, about 3 in³, about 4 in³, about 5 in³, about 6 in³, about 7 in³, about 8 in³, about 9 in³, about 10 in³, about 11 in³, about 12 in³, about 13 in³, about 14 in³, about 15 in³, about 20 in³, about 25 in³, or about 30 in³.

Also provided herein is a method of ventilating an orthopedic cast comprising: (a) inserting a flexible tubing into the space between the orthopedic cast and the body of the wearer of the orthopedic cast, wherein the flexible tubing is connected to an air-pump and comprises an opening through which air can be released from the flexible tubing into the space between the orthopedic cast and the body of the wearer of the orthopedic cast; and, (b) operating the air-pump to release air into the flexible tubing, thereby ventilating the space between the orthopedic cast and the body of the wearer of the orthopedic cast.

### EXAMPLES

### Example 1

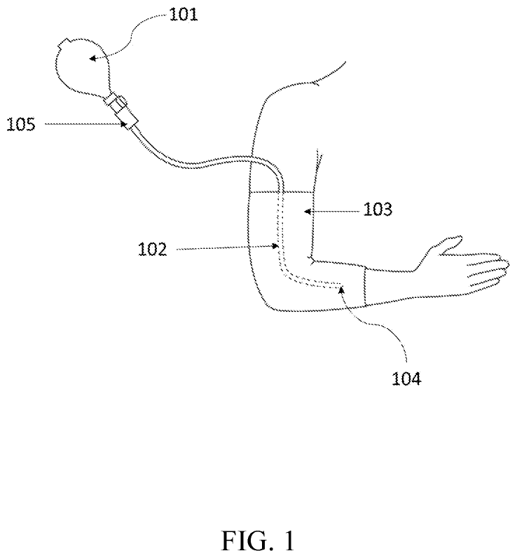

FIG. 1 shows a device that permits ventilation of an orthopedic cast. This device was made by connecting a manually operable air-pump, 101, to a plastic tubing, 102. The air-pump was connected to the plastic tubing using a plastic connector, 105. The device was then used to ventilate a cast, 103. This device had one opening, 104 at the end of the plastic tubing. In the figure, the exemplified cast is around the arm of the wearer, but it could be applied to a cast encasing any other body part. Adequate air was found to be released into the space between the orthopedic cast and the body of the wearer of the orthopedic cast by manually operating the air-pump, thereby ventilating and cooling the cast.

### Example 2

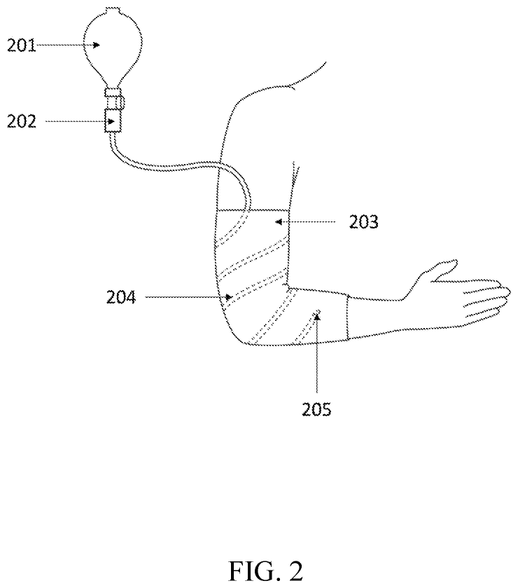

FIG. 2 shows a device for ventilating an orthopedic cast where the flexible tubing is configured such that it can be wrapped around the limb of wearer in a helical manner. A manually operable air-pump, 201, is connected to a flexible tubing, 204 through a connector 202. The device can then be used to ventilate a cast, 203. The exemplified device has an opening, 205, at the end of the tubing through which air can be pushed out into the cast. But, the device can also be configured to have multiple openings along the flexible tubing inside the cast.

### Example 3

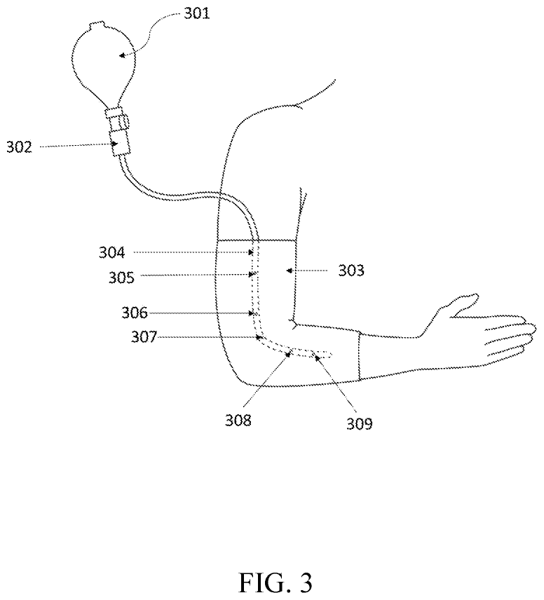

FIG. 3 shows a device for ventilating orthopedic cast that permits ventilation of the orthopedic cast through multiple openings in the flexible tubing. This device includes a manually operable air-pump, 301 connected to a flexible tubing, 304. The air-pump is connected to the flexible tubing using a connector, 302. The device can then used to ventilate a cast, 303. This device has multiple openings, 305-309 in the flexible tubing. In the figure, the exemplified cast is around the arm of the wearer, but it could be applied to a cast encasing any other body part.

The examples disclosed in this application are to be considered in all respects as illustrative and not limiting. The scope of the invention is indicated by the appended claims rather than by the foregoing description; and all changes which come within the meaning and range of equivalency of the claims are intended to be embraced therein.

1. A device for ventilating an orthopedic cast comprising:
(a) an air-pump; and
(b) a flexible tubing connected to the air-pump;
wherein the flexible tubing is insertable into the space between the orthopedic cast and the body of the wearer of the orthopedic cast;
wherein the air-pump permits air to be forced into the flexible tubing; and
wherein the flexible tubing comprises an opening that permits the air to ventilate the space between the orthopedic cast and the body of the wearer of the orthopedic cast.

2. The device of claim 1, wherein the flexible tubing comprises more than one opening that permits the air to be released into the space between the orthopedic cast and the body of the wearer of the orthopedic cast.

3. The device of claim 1, wherein the flexible tubing is connected to the air-pump via a connector.

4. The device of claim 1, wherein the connector is a tubing of larger diameter than the flexible tubing.

5. The device of claim 1, wherein the air-pump can be operated manually.

6. The device of claim 1, wherein the flexible tubing is made of biocompatible material.

7. The device of claim 6, wherein the biocompatible material is a biocompatible plastic.

8. The device of claim 1, wherein the flexible tubing has a diameter of about 0.1 inch to about 0.5 inch.

9. The device of claim 1, wherein the flexible tubing has a diameter of about 0.2 inch to about 0.3 inch.

10. The device of claim 1, wherein the flexible tubing has a diameter of about 0.25 inch.

11. The device of claim 1, wherein the air-pump permits adjusting the pressure of the air forced into the flexible tubing.

12. The device of claim 1, wherein the device is attached to the orthopedic cast.

13. The device of claim 1, wherein the flexible tubing is attached to the orthopedic cast.

14. A method of ventilating an orthopedic cast comprising:
(a) inserting a flexible tubing into the space between the orthopedic cast and the body of the wearer of the orthopedic cast,
wherein the flexible tubing is connected to an air-pump and comprises an opening through which air can be released from the flexible tubing into the space between the orthopedic cast and the body of the wearer of the orthopedic cast; and,
(b) operating the air-pump to release air into the flexible tubing, thereby ventilating the space between the orthopedic cast and the body of the wearer of the orthopedic cast.

15. The method of claim 14, wherein the flexible tubing comprises more than one opening that permits the air to be released into the space between the orthopedic cast and the body of the wearer of the orthopedic cast.

16. The method of claim 14, wherein the flexible tubing is connected to the air-pump via a connector.

17. The method of claim 14, wherein the air-pump can be operated manually.

18. The method of claim 14, wherein the flexible tubing is made of biocompatible material.

19. The method of claim 14, wherein the flexible tubing has a diameter of about 0.1 inch to about 0.5 inch.

20. The method of claim 14, wherein the air-pump permits adjusting the pressure of the air forced into the flexible tubing.
